(19) United States
(12) Patent Application Publication
(10) Pub. No.: US 2008/0223380 A1
(43) Pub. Date: Sep. 18, 2008

(54) Tissue protective system and method for thermoablative therapies

(76) Inventor: Douglas O. Chinn, Arcadia, CA (US)

Correspondence Address: Morland C. Fischer, Suite 1300, 2030 Main Street, Irvine, CA, 92614, US

(21) Appl. No.: 12/154,545

(22) Filed: May 22, 2008

Related U.S. Application Data
(62) Division of application No. 11/003,971, filed on Dec. 6, 2004.

(51) Int. Cl. A61B 18/04; A61B 18/18; A61F 7/00; A61F 7/12

(52) U.S. Cl. 128/898; 606/27; 606/41; 607/105; 607/113

(57) ABSTRACT

A tissue protective system and method having particular application in thermoablative surgical therapies where heat or cold is used to create a kill zone for treating cancer cells as well as malignant or benign tumors in a targeted internal tissue area (e.g., the prostate) of a patient while sparing an adjacent benign internal tissue area (e.g., a neurovascular bundle). One of a hollow sheath or a balloon that is carried by a balloon catheter is located within an access opening that is made by a needle trocar inserted between the targeted tissue area in need of treatment and the benign tissue area to be protected in order to hold the protected tissue area off the targeted tissue area and away from the lethal temperature of the kill zone. The balloon of the balloon catheter is inflated in the access opening via a balloon channel which runs longitudinally through the catheter. At least one temperature sensor is mounted on the balloon and responsive to the temperature near the benign tissue area to be protected. Heat or cold is provided to the balloon from a heating wire or a circulating fluid, depending upon the temperature that is sensed by the temperature sensor.

### BACKGROUND OF THE INVENTION

1. Field of the Invention

This invention relates primarily to a balloon catheter associated with a temperature monitoring and control device and being adapted to protect (e.g., dissect, insulate, heat or cool) a defined internal tissue area to be spared that is located adjacent to a targeted internal tissue area which undergoes minimally invasive thermoablative therapy.

2. Background Art

In the treatment of benign and malignant conditions, minimally invasive therapies have been used in the past and are currently being developed today. These therapies are usually thermoablative in nature and include cryosurgery, high frequency ultrasound, thermomagnetic, microwave, and radio frequency therapies. Newly developed thermoablative therapies typically require a percutaneous access to an internal area of the body requiring treatment. Such percutaneous access is guided by imaging technology, i.e., CT scan, MRI, X-ray, ultrasound and other developing modalities. In the broadest sense, and despite the current therapies, there is tissue surrounding the targeted organ or tissue of the patient in need of treatment that cannot or should not be damaged by the therapy. Such delicate organs/tissue which should be protected during percutaneous access include liver, renal, uterine and prostate regions when tumors are to be treated.

For example, in the case of prostate cancer, all thermoablative technologies typically ablate the entire prostate gland. An undesirable side effect of total gland ablation is injury or destruction to one or both of the neurovascular bundles (NVBs) which run bilaterally on the surface of the gland in a posterior lateral position. The neurovascular bundles are required for patients to attain a spontaneous erection. If such bundles are damaged, injured or accidentally removed during prostate cancer treatment, the risk of male impotency is increased. Because of this risk, many male patients do not seek screening for prostate cancer, delay definitive therapy, or attempt holistic therapy with the hopes of avoiding impotency as a side effect of the treatment. This delay in diagnosis or avoidance of proper treatment can often lead to continued growth and advancement of the cancer until an incurable stage is reached.

If there is a large volume of cancer or the cancer appears to have penetrated through the capsule of the prostate gland, then destruction of the NVB often becomes necessary. With surgical extirpation (i.e., radical prostatectomy) of the prostate gland under ideal conditions, whether by open or laparoscopic radial prostatectomy, the surgeon has attempted to save one or both of the patient's NVBs. However, during the ablative surgical procedure, it is often difficult to dissect or otherwise lift the NVB off the gland which, consequently, results in an injured NVB. In order to otherwise avoid injuring the NVBs during ablative therapy, a portion of the prostate gland may have to remain untreated, which potentially leaves some of the cancer behind.

One widely accepted form of thermoablative therapy which relies on cold to treat prostate cancer is cryosurgery. Localized heating is another form of thermoablative therapy which relies on heat treatment. Unfortunately, the conventional cryosurgical and heating techniques have proven to be flawed, such that the heating might be overcome by the freezing probes or be too strong and thereby damage the NVB by excess heat. A cooling method is required for ferromagnetic alloy implants and high frequency ultrasound (HIFU), inasmuch as these two treatments are based upon heat to ablate the tissue. Radio frequency and microwave ablative therapy are similarly based upon heat and also require a suitable cooling method. Once again, however, there is either too little cooling and the NVBs are damaged or destroyed or there is too much cooling and cancerous tissue may be left behind. The reason tissue is usually left behind is based upon a thermal gradient that occurs with all thermoablative therapy. That is, the coldest or hottest temperatures occur immediately adjacent the thermal device and decrease with distance. There is a target tissue temperature for either heating or cooling ablative temperature that must be achieved to successfully destroy a cancerous tumor. However, it has proven to be difficult to precisely control the heating and cooling down to the precise millimeters of the tissue requiring treatment.

### SUMMARY OF THE INVENTION

Briefly, and in general terms, disclosed herein are a tissue protecting and sparing method and a balloon catheter having particular application for use in the treatment of prostate (or other organ) cancer by means of thermoablative therapy. In those cases where a minimally invasive procedure is desirable, the prostate gland is either frozen or heated to a lethal temperature (e.g., by means of cryoablating the prostate with iceballs or by means of microwave, thermomagnetic radio frequency, or high frequency ultrasound treatments). According to the tissue sparing method of this invention, a balloon catheter is inserted in a channel that is formed between a patient's prostate to be treated and the neurovascular bundle (NVB) to be protected. When the balloon of the catheter is inflated, the patient's NVB is correspondingly lifted off and dissected from the prostate gland undergoing treatment. Thus, not only will the inflated balloon function as a thermal insulator and reflector of soundwaves, but the NVB will be spared from the lethal temperature to which the prostate gland is frozen or heated. In the alternative, the tissue sparing method of this invention can also be achieved by means of a sheath that is carried by a diamond tipped needle trocar. The trocar cuts an access channel through the patient's tissue and is then withdrawn leaving the sheath behind to provide separation and thermal isolation between the patient's prostate and the NVB. By virtue of the inflated balloon and the sheath, the NVB will be protected from possible removal or damage which has been known to result in the patient becoming sexually impotent.

The aforementioned balloon catheter includes a pair of temperature sensors (e.g., T-type thermocouples) that are fused to opposite sides of the balloon. Electrical wires extend from the temperature sensors to a temperature monitor and control device. The pair of temperature sensors are positioned on opposite sides of the balloon so that one sensor is responsive to the temperature of the targeted prostate gland undergoing thermoablative treatment, and the second sensor is responsive to the temperature of the NVB to be protected. In the event that the temperature of the NVB begins to approach the fatal temperature at the kill zone, the temperature monitor and control device is adapted to generate a feedback signal by which to cause the NVB to automatically receive a supply of heat or cold in addition to the insulating effect produced by the balloon.

According to a first catheter embodiment, a heating wire runs longitudinally through the shaft of the catheter to be surrounded by the balloon. The balloon is inflated with air, or the like. The feedback signal generated by the temperature monitor and control device is applied to a wire heater by which to energize the heating wire and enable the inflated balloon to be heated. According to a second catheter embodiment, the feedback signal generated by the temperature monitor and control device is applied to a fluid heater/cooler which is coupled to a fluid pump in a fluid circuit. A heated or cooled fluid (e.g., water or gas) is continuously circulated through the catheter to cause the balloon to inflate while being heated or cooled. So long as the temperature monitor and control device receives an indication from the temperature sensors that the temperature of the NVB to be protected has been raised or lowered to a safe, non-lethal level, an additional feedback signal is generated by which to deactivate the wire heater or the fluid heater/cooler.

### BRIEF DESCRIPTION OF THE DRAWINGS

FIG. 1A is an anatomical view showing a needle trocar located in an access channel between a patient's neurovascular bundle to be protected and spared and the prostate gland in need of thermoablative therapy to a lethal temperature;

FIG. 1B shows the anatomical view of FIG. 1A with a balloon catheter positioned within the channel formed by the needle trocar between each of the patient's neurovascular bundles and the prostate gland to which cryoablative treatment is to be applied;

FIG. 1C shows the anatomical view of FIG. 1B for cryoablating the patient's prostate gland in need of treatment by means of iceballs with balloon catheters holding the neurovascular bundles off the prostate;

FIG. 2 shows a balloon catheter according to a first embodiment which can be located in the access channel of FIG. 1A between the patient's neurovascular bundle and the prostate gland;

FIG. 2A shows a balloon catheter according to a second embodiment which can also be located in the access channel of FIG. 1A;

FIG. 3 is a cross-section of the balloon catheter taken along lines 3-3 of FIG. 2;

FIG. 4 illustrates a temperature monitoring and control system by which the balloon catheters of FIGS. 2 and 2A are adapted to receive heat or cold to be applied to the patient's neurovascular bundle depending upon the temperature of the prostate gland during thermoablative therapy relative to the temperature of the neurovascular bundle;

FIG. 5 shows an exploded view of a percutaneous access system that is used to form the access channel between the patient's neurovascular bundle to be spared and the prostate gland to which cryoablative treatment is to be applied;

FIG. 6 shows the percutaneous access system of FIG. 5 as it will be assembled and installed to form the access channel; and

FIG. 7 shows a sheath from the percutaneous access system of FIGS. 5 and 6 according to another preferred embodiment for use as an alternative to the balloon catheters of FIGS. 2 and 2A for holding the patient's neurovascular bundles off the prostate during cryosurgery.

### DESCRIPTION OF THE PREFERRED EMBODIMENT

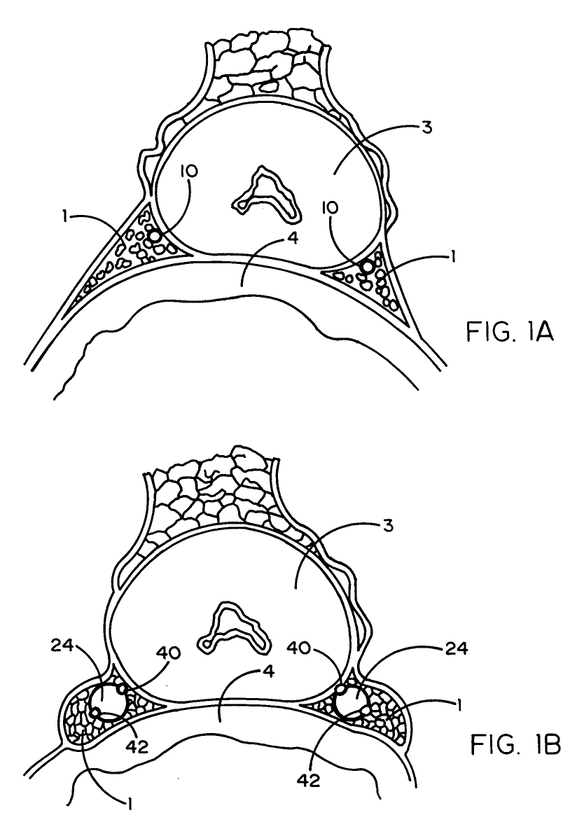

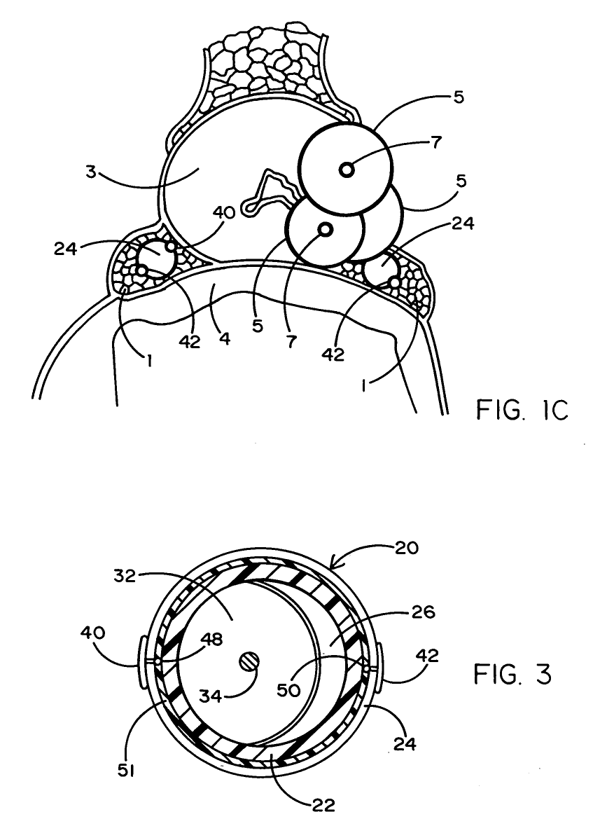

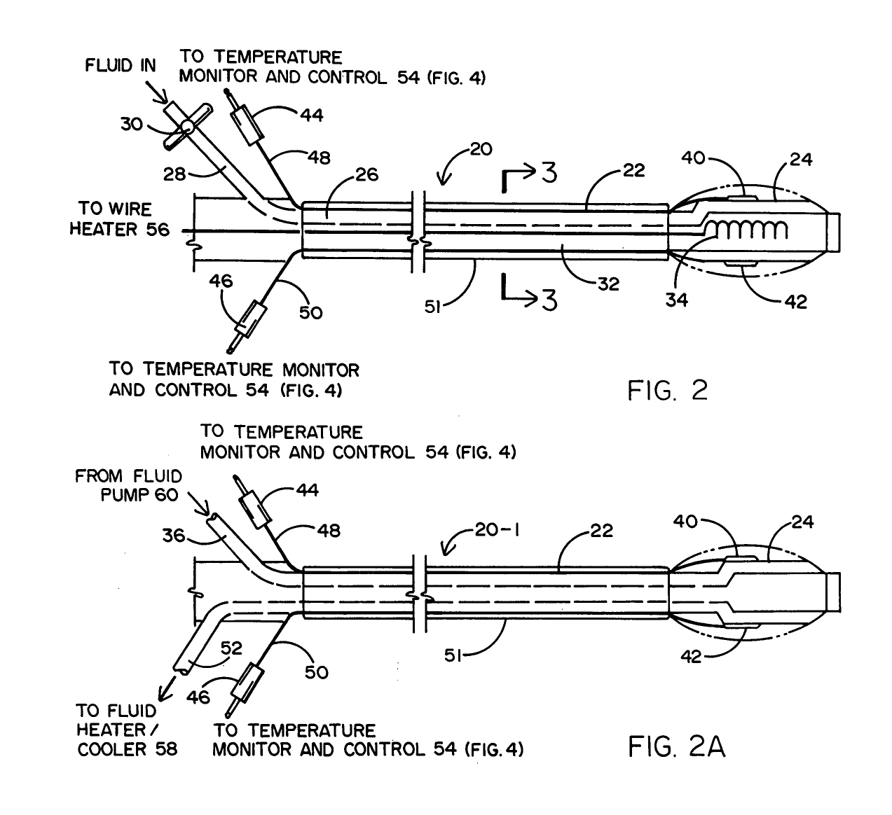

Referring to the drawings, a portion of the human anatomy is illustrated in FIG. 1A to show the neurovascular bundles (NVBs) 1 located above the rectum 4 and attached to opposite sides of the prostate gland 3 in need of treatment for cancer. In order to preserve the NVBs 1 during a thermoablative surgical procedure, they must be moved off or dissected from the prostate gland 3. As will be explained in greater detail hereinafter, and as an important improvement to conventional minimally invasive thermoablative techniques, unique balloon catheters (designated 20 and 20-1 in FIGS. 2 and 2A) have a balloon 24 that is inflated between each of the patient's NVBs 1 and the prostate gland 3 so as to elevate, separate and spare the NVB off the prostate. By preserving and sparing the NVBs 1, sexual potency of the patient can be maintained following surgery. That is, the patient could be made impotent if the NVBs were to be surgically removed or damaged during thermal (or mechanical) ablation.

What is more, and as will also be explained, in order to freeze cancerous tissue of the prostate gland 3 to a lethal temperature during cryosurgery while the NVBs 1 will be protected, the balloon catheters 20 and 20-1 of this invention are advantageously provided with temperature sensing and control means. By monitoring the temperature at the kill zone and near the NVBs, a determination can be made as to whether the NVBs will receive the same lethal temperature to which the cancerous prostate tissue is subjected during cryosurgery. Should this be the case, then heat may be generated to protect the NVBs from reaching a potentially fatal temperature.

In FIG. 1A, a diamond tipped needle trocar 10 is used according to a well known Seldinger method in order to form access channels medial to each of the patient's neurovascular bundles to accommodate one of the balloon catheters 20 or 20-1 of FIGS. 2 and 2A) so that the balloon 24 thereof can be inflated between the prostate gland 3 requiring thermoablative treatment and the benign neurovascular bundles 1 to be spared in the manner shown in FIG. 1B. Once the trocar 10 is properly positioned, a stiff J-tipped guidewire is passed through the trocar 10 and secured by its tip. Under ultrasound guidance, the balloon catheter is passed over the guidewire. When the catheter is moved between the neurovascular bundle and the prostate gland, the guidewire is removed and the balloon 24 is inflated. By virtue of the foregoing, the neurovascular bundles will be preserved while the entire prostate can still be engulfed with iceballs in the manner shown in FIG. 1C.

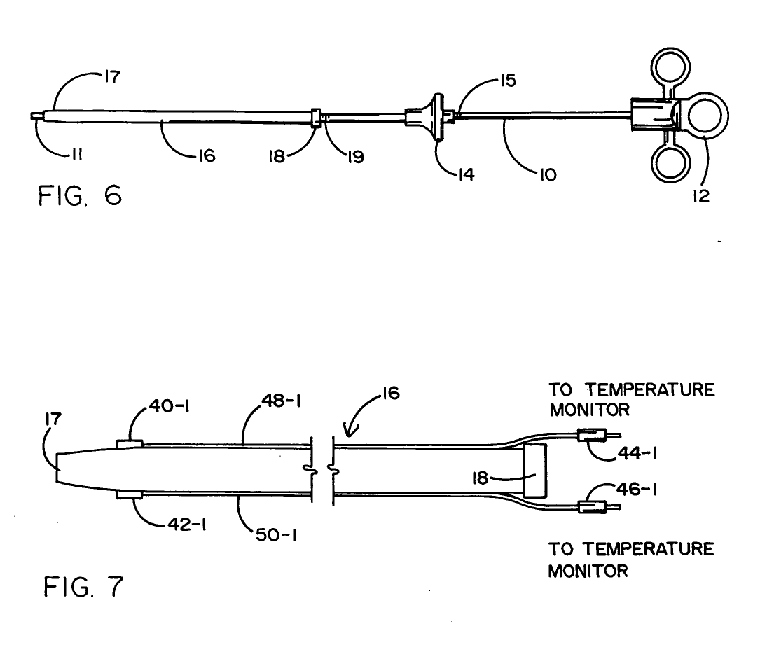

Referring briefly to FIGS. 5-7 of the drawings, a percutaneous access system is described to facilitate an alternate method for sparing the patient's NVBs during cryosurgery. The access system includes a (e.g., 30 cm long) needle trocar 10 having a diamond tip 11 at one end that is adapted to cut an access channel through the patient's tissue. A handle 12 having thumb and finger holes is located at the opposite end of needle trocar 10. The access system also includes a standard tapered (e.g., 22.5 cm long) dilator 13 having a central channel that is sized to fit snugly over the needle trocar 10. The rear end of dilator 13 has a leur lock fitting 14 to hold the dilator in place in surrounding engagement with needle trocar 10. Visible markings (designated 15 in FIG. 6) are made on the needle trocar 10 to enable the surgeon to know when the dilator 13 has reached the distal tip 11 of the trocar during installation of the access system as will soon be described. The access system also includes a (e.g., 16 cm long) outer sheath 16 having a tapered distal tip 17 located at one end and a flange 18 surrounding the opposite end to be gripped by the surgeon. The outer sheath 16 is sized to fit over the dilator 13. Visible markings (designated 19 in FIG. 6) are made on the dilator 13 to enable the surgeon to know when the sheath 16 has reached the distal tip of the dilator 13 during installation of the access system as will soon be described.

To make an access channel through the patient's tissue, the percurtaneous access system comprising needle trocar 10, dilator 13, and outer sheath 16, is first assembled one over the other in the manner shown in FIG. 6. Ultrasound, color flow Doppler, or any other suitable imaging modality can be used to assist the surgeon during placement of the diamond tipped needle trocar 10 between the prostate and the neurovascular bandle. The needle trocar 10 is advanced through the patient's tissue until the dilator 13 which surrounds the trocar reaches the patient's skin. Next, the skin is incised to allow the dilator 13 to penetrate the patient's tissue. The percutaneous access system is now further advanced until its final position is reached.

At this point, by using the markings 15, the dilator 13 is moved down to the tip 11 of the trocar 10 (best shown in FIG. 6). By using the markings 19, the outer sheath 16 is moved down to the tip of the dilator 13 (also best shown in FIG. 6). Finally, the needle trocar 10 and the dilator 13 are removed leaving the sheath 16 in place. The balloon catheter 20 or 20-1 (of FIGS. 2 and 2A) is then advanced through the sheath 16 with the balloon 24 thereof in an uninflated condition. The sheath 16 is partially retracted along the catheter so as to move past and out of the way of the balloon 24 to allow its inflation.

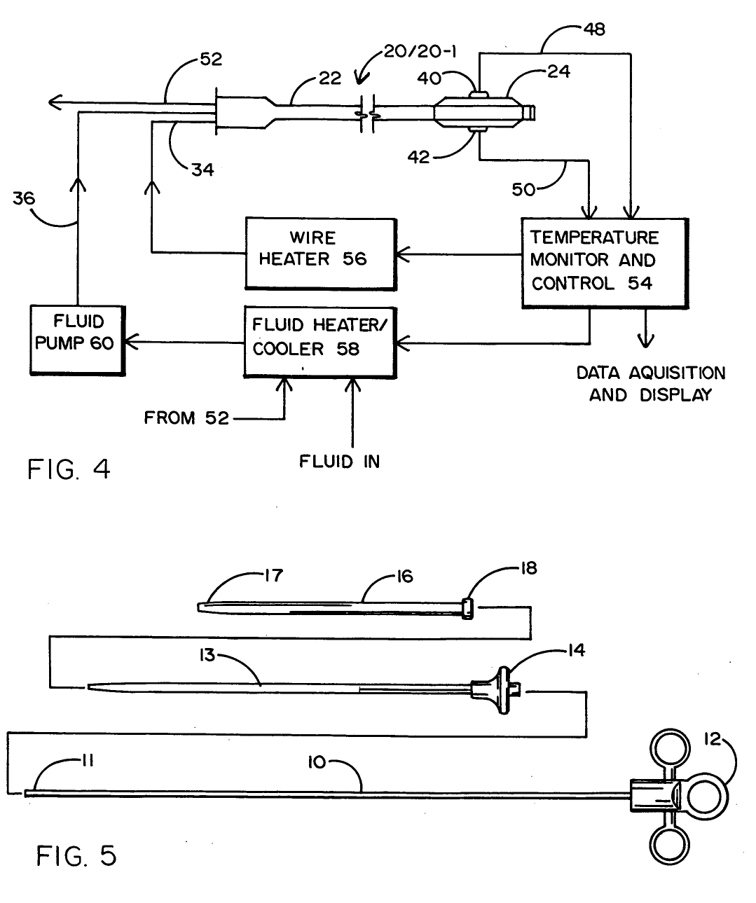

Once the balloon 24 of the catheter 20 or 20-1 is inflated, the NVB 1 will be correspondingly lifted off and pushed away from the prostate gland 3 as shown in FIG. 1B. In a first preferred embodiment, temperature sensors 40 and 42 are mounted on opposite sides of the balloon 24 whereby temperature information can be supplied to a temperature monitor and control device (designated 54 in FIG. 4) so that a feedback control signal is generated if the NVB 1 will experience a temperature that approaches a predetermined lethal temperature at which the prostate gland will be cryoablated.

For cryosurgery, air can be used to inflate the balloon 24 of the catheter 20 of FIG. 2 as well as to function as an insulator between the NVB and the lethal temperature to which the prostate gland is frozen. If air alone is insufficient, then a heating wire (designated 34 in FIGS. 2 and 3) running through the catheter 20 can be energized to heat the air surrounding the balloon 24. If the heated air still will be insufficient to protect the NVBs, then the catheter 20-1 of FIG. 2A can be used to receive a heated fluid. At the conclusion of the surgery, the balloon 24 will be deflated and withdrawn through the sheath 16. The sheath 16 is then withdrawn from the patient's tissue.

According to another preferred embodiment, the tissue sparing advantages of this invention can be achieved without the introduction of the balloon catheter 20 or 20-1 through the sheath 16. In this case, the sheath 16, alone, will provide separation and insulation between the prostate 3 or other targeted tissue area to be treated and the NVB 1 or other tissue area to be protected. FIG. 7 of the drawings shows details of the sheath 16 from the percutaneous access system of FIGS. 5 and 6 being used in place of the balloon 24 of the balloon catheter 20.

Like the balloon 24, the sheath 16 of FIG. 7 includes a pair of temperature sensors 40-1 and 42-1 (e.g., conventional thermistors or T-type thermocouples) so that temperature information can be supplied to a temperature monitor (not shown) to enable the surgeon to determine if the patient's NVB will be exposed to the same potentially fatal temperature at which the prostate gland is frozen during surgery. The temperature sensors 40-1 and 42-1 are mounted about 1 cm from distal tip 17 and fused on opposite sides of the sheath 16 so as to lie in thermal contact with the prostate gland to be treated and the NVB to be spared. The temperature sensors 40-1 and 42-1 are connected to respective electrical plugs 44-1 and 46-1 by means of flexible electrical conductors 48-1 and 50-1 that are bonded to and run longitudinally along the sheath 16. The conductors 48-1 and 50-1 may be covered by a sleeve (not shown) along sheath 16. The plugs 44-1 and 46-1 that are coupled to temperature sensors 40-1 and 42-1 are connected to the aforementioned temperature monitor to provide a warning to the surgeon that the temperature at the NVB must be raised to avoid possible injury.

It is to be understood that the improvements disclosed herein are not limited to cryosurgery. Such improvements are particularly applicable to any prostate (or other targeted organ) therapy that is minimally invasive and the ablation is based upon heat, cold, microwave, thermomagnetic, radio frequency, high frequency ultrasound, or the like, where tissue sparing is of paramount importance. In fact, the advantages of this invention can also be extended to open or laparoscopic radial prostatectomies.

In this same regard, for heated thermoablative treatments, cooling could be used to prevent the NVBs from reaching a fatal temperature. In this case, thermoelectric energy, air and/or water can be employed to protect the benign NVB tissue. For high frequency ultrasound treatment, simply lifting the NVBs 1 off the prostate gland 3 coupled with the reflective properties of the inflated balloon 24 will typically prevent the sound waves from damaging the NVBs. For radial prostatectomy, either the sheath 16 or the balloon catheter can be used in the process of dissecting the NVBs off the prostate gland.

Once the NVBs are lifted off the prostate gland 3 by means of the balloon catheter 20 or 20-1 of FIGS. 2 and 2A or by the sheath 16 of FIGS. 5 and 6, ice balls (designated 5 in FIG. IC) can be formed at the tips of respective cryoprobes 7 so as to engulf the prostate gland under treatment. Accordingly, the entire gland 3 can now be cryoablated with the inflated balloon or sheath pushing each NVB 1 away from the lethal ablation, whereby to avoid injuring the NVB and adversely affecting the patient's potency. Reference can be made to my earlier U.S. Pat. No. 5,647,868 issued Jul. 15, 1997 for a more complete teaching of a method and system for cryoablating the prostate gland by means of iceballs.

Turning now to FIGS. 2 and 3 of the drawings, there is shown the details of one balloon catheter 20 according to the first preferred embodiment having an elongated, flexible shaft 22 and the aforementioned balloon 24 wrapped around the distal end of the shaft 22 in an uninflated condition. The catheter 20 is approximately 22 cm in length, while the balloon 24 is approximately 4 cm long. For most thermoablative application, the balloon 24 should have a burst pressure of at least 20 ATM. The balloon 24 is preferably located approximately 5 mm from the distal end of the catheter shaft 22. The outside diameter of balloon 24 in its uninflated state is approximately 5 mm. Note that these dimensions and parameters are ideal, but are not intended to limit the scope of my invention.

A relatively narrow balloon channel 26 runs longitudinally through the shaft 22 of catheter 20. One end of balloon channel 26 (best shown in FIG. 3) communicates with the balloon 24. The opposite end of balloon channel 26 communicates with a balloon port 28 that is adapted to be connected to a source of fluid (e.g., water, air or any other suitable gas) by way of a suitable inflation device (e.g., inflation set No. CIDS-25 manufactured by Cook Medical, Inc.). A manually rotatable stopcock valve 30 is associated with the balloon port 28 to close and open port 28 and thereby block or permit the delivery of fluid from the source thereof to the balloon 24 via balloon channel 26 depending upon the position of valve 30. Once the catheter 20 has been inserted in the channel formed between the patient's neurovascular bundle and the prostate gland under treatment and the stopcock valve 30 is rotated to the open position, fluid will be delivered to the balloon channel 26, and the balloon 24 will be inflated as shown in phantom lines in FIG. 2. Accordingly, the patient's neurovascular bundle 1 will be pushed off the prostate gland 3 in the manner illustrated in FIG. 1B.

Also running longitudinally through the shaft 22 of catheter 20 alongside the balloon channel 26 is a working channel 32 (also best shown in FIG. 3). So as to be able to provide a supply of heat in order to prevent the lethal temperature of the prostate gland under treatment in the kill zone from possibly reaching and freezing the patient's neurovascular bundle to a fatal temperature, one end of a (e.g., resistance) heating wire 34 is slid down the working channel 32 so as to be surrounded by the balloon 24. The opposite end of the heating wire 34 is connected to a wire heater (designated 56 in FIG. 4) so that a controlled heat can be selectively generated and provided to the balloon 24 depending upon the temperatures that are detected by the temperature sensors carried at opposite sides of the balloon 24 in a manner that will now be described.

In order to avoid damage to the patient's neurovascular bundles near the kill zone in which the prostate is being treated by means of cryoablation, it is important to be able to monitor and control the temperature to which the neurovascular bundles will be exposed. To accomplish the foregoing, a pair of temperature sensors 40 and 42 are mounted at opposite sides and near the middle of the balloon 24 of catheter 20. By way of example only, the temperature sensors 40 and 42 may be conventional thermistors or T-type thermocouples. Each temperature sensor 40 and 42 is connected to a respective electrical plug 44 and 46 by means of a flexible electrical conductor 48 and 50 that is bonded to and runs longitudinally along the shaft 22 of catheter 20. The conductors 48 and 50 are preferably covered by a sleeve 51 along the shaft 22. However, the conductors 48 and 50 must remain free floating (i.e., not bonded) relative to balloon 24 to compensate for the inflation thereof. The plugs 44 and 46 that are coupled to the temperature sensors 40 and 42 at opposite sides of balloon 24 are connected to a temperature monitor and control device (designated 54 in FIG. 4).

Referring briefly once again to FIG. 1B of the drawings, an inflated catheter balloon 24 of balloon catheter 20 is shown located in a channel that is formed through the patient's tissue in the manner earlier described at each side of the prostate gland 3 to lift the patient's neurovascular bundles 1 off the prostate and away from the lethal temperature of the freezing zone. The pair of temperature sensors 40 and 42 are preferably fused (e.g., acoustically welded) to opposite sides of balloon 24 so that one temperature sensor 40 will be responsive to the temperature at which the targeted prostate gland under treatment is frozen while the opposing temperature sensor 42 will be responsive to the temperature to which the benign neurovascular bundle to be preserved is exposed. However, and as indicated above, the temperature sensors 40 and 42 may also be responsive to heat should the prostate gland receive treatment that is based upon a thermal ablative therapy using heat, microwaves, thermomagnetics, radio frequency, high frequency ultrasound, etc.

By monitoring and comparing the temperature at the kill zone during cryoablative therapy and the temperature near the patient's neurovascular bundles to be spaced and insulated from the kill zone, an indication will be available should the temperature of the neurovascular bundles approach a potentially fatal temperature. In this case, a feedback signal is provided from the temperature monitor and control device (54 of FIG. 4) to the wire heater (56 of FIG. 4) by which to automatically cause the heating wire 34 that runs longitudinally through the working channel 32 to the balloon 24 of catheter 20 to be heated. Accordingly, the heating wire 34 surrounded by the balloon 24 will generate a controlled heat in order to raise the temperature of the air surrounding balloon 24 and thereby protect the neurovascular bundles. Once the temperature of the air around the neurovascular bundles has been elevated to a predetermined safe level and the corresponding temperature difference detected by sensors 40 and 42 is increased, another feedback signal will be provided from the temperature monitor and control device 54 to the wire heater 56 to deenergize the heating wire 34 and thereby end the heating process.

In the simplest form of this invention, the balloon catheter 20 will be devoid of the heating wire 34 running therethrough. In this case, the balloon 24 is merely filled with air which would then function to insulate the neurovascular bundles to be protected from the lethal temperature of the kill zone and/or to reflect sound waves. Alternatively, the heating wire 34 can be replaced by a thermoelectric cooling wire (not shown) running through the working channel 32 of catheter 20 for heat therapy applications. Such a thermoelectric cooling wire will carry a series of thermoelectric cooling elements (sometimes known as Peltier voltage controlled heat exchange devices) by which to cool the area surrounding the balloon 24.

FIG. 2A of the drawings shows a second balloon catheter 20-1 for use in thermoablative applications where the heating wire 34 of the balloon catheter 20 of FIG. 2 is not adequate, desirable or available. In this case, the heating wire 34 of catheter 20 is replaced in catheter 20-1 by a fluid circuit including a fluid heater/cooler (designated 58 in FIG. 4) and a fluid pump (designated 60 in FIG. 4). Identical reference numbers are used to designate features of the balloon catheter 20-1 of FIG. 2A which are the same as the features of balloon catheter 20 of FIG. 2 and, therefore, the details of such common features will not be described once again.

In the balloon catheter 20-1 of FIG. 2A, water or any other suitable fluid or gas (e.g., saline solution) is heated or cooled by the fluid heater/cooler 58 of FIG. 4 and pumped down a longitudinally extending fluid inlet 36 through the shaft 22 of catheter 20-1 by means of fluid pump 60. The fluid inlet 36 communicates with the balloon 24 of catheter 20-1, whereby the fluid carried thereby causes the balloon to inflate. A fluid outlet or return 52 also communicates with the balloon 24 and runs longitudinally through the shaft 22 of catheter 20-1 to the fluid heater/cooler 58 (best shown in FIG. 4). By virtue of the foregoing, a heated or cooled fluid can be continuously circulated through catheter 20-1 to provide a controlled heating or cooling to the inflated balloon 24 (depending upon the temperature signals supplied by sensors 40 and 42 to the temperature monitor and control device 54 of FIG. 4) to prevent the patient's neurovascular bundle from reaching a potentially lethal temperature.

Turning specifically now to FIG. 4 of the drawings, there is shown a system diagram of the balloon catheters of my invention and the pair of temperature sensors 40 and 42 located at opposite sides of balloon 24 and connected to the temperature monitor and control device 54 so that a feedback signal can be generated for controlling the operation of the wire heater 56 of the catheter 20 of FIG. 2 or the fluid heater/cooler 58 of the catheter 20-1 of FIG. 2A. That is, and as was earlier disclosed, in the event that the temperature to which either one or both of the patient's neurovascular bundles to be protected should approach a potentially fatal temperature near the lethal temperature at the kill zone during cryoablative or heat therapy, a controlled supply of heat or cold is generated at the interior of balloon 24 so as to raise or lower the temperature of the neurovascular bundles to a safe level. To this end, the maximum safe temperature to which the patient's neurovascular bundle can be safely exposed is preprogrammed into the temperature monitor and control device 54. What is more, the temperature monitor and control device 54 can be linked to a computerized real time data acquisition and display system (similar to that described in my earlier U.S. Pat. No. 5,647,868) so that the temperatures detected by temperature sensors 40 and 42 and the difference therebetween can be recorded and/or displayed to the surgeon.

In one embodiment, the wire heater 56 of the balloon catheter 20 of FIG. 2 energizes the heating wire 34 running through the working channel 32 to the balloon 24. In another embodiment, without a heating wire, the fluid heater/cooler 58 and pump 60 are connected in a fluid circuit with the balloon catheter 20-1 of FIG. 2A to cause either a heated or cooled fluid (i.e., a liquid or a gas) to be circulated through the balloon 24. The wire heater 56 and the fluid heater/cooler 58 will be disabled for as long as the temperature sensors 40 and 42 indicate that the neurovascular bundles are no longer in danger of approaching the potentially fatal low or high temperature of the kill zone. By virtue of the foregoing, the temperature in and around the patient's neurovascular bundle can be maintained at a predetermined non-fatal level in order for the neurovascular bundle to be spared from being frozen (or heated) while freezing (or heating) the entire prostate gland in need of thermoablative therapy to a lethal temperature.

The balloon catheters 20 and 20-1 of FIGS. 2 and 2A and the sheath 16 of the precutaneous access system of FIGS. 5-7 have been advantageously used in a unique application to lift a patient's neurovascular bundles off the prostate gland during thermoablative therapy in order to protect and spare the neurovascular bundles from reaching a potentially fatal temperature and thereby avoiding the corresponding damage as a consequence thereof. In this same regard, the sheath 16 and the balloon catheters 20 and 20-1 herein disclosed as well as the tissue sparing method for which the sheath and catheters are used are not limited solely to treatment of the prostate gland while protecting the adjacent benign neurovascular bundles. More particularly, the sheath 16, the balloon catheters 20 and 20-1, and the tissue sparing method disclosed herein are also applicable to other targeted organs and tissue in need of thermoablative therapy. By way of example only, a lesion may be formed on the kidney, and it may be desirable to protect and hold the adjacent intestine off the kidney during treatment. A lesion formed on the liver, breast, uterus, renal gland, etc. may also require treatment at the same time that it is desirable to spare the benign tissue adjacent thereto.

I claim:

1. A surgical method for moving a benign internal tissue area of a patient to be protected away from an adjacent targeted internal tissue area to be treated with thermoablative therapy by means of cooling or heating the targeted tissue area to a lethal temperature, said method comprising the steps of:
forming an access channel between the targeted internal tissue area to be treated and the adjacent benign internal tissue area to be protected;
locating within the access channel a balloon catheter having an uninflated balloon; and
inflating the balloon of the balloon catheter so as to spare the benign internal tissue area to be protected from the lethal temperature to which the targeted internal tissue area is cooled or heated.

2. The method recited in claim 1, wherein the balloon of the balloon catheter is filled with air during said inflating step, whereby said air filled balloon forms a thermal insulator between said benign tissue area to be protected and said targeted tissue area to be treated.

3. The method recited in claim 1, including the additional step of heating or cooling the inflated balloon of the balloon catheter and correspondingly raising or lowering the temperature of the benign tissue area to be spared from the lethal temperature of the targeted tissue area, depending upon whether the targeted tissue area is treated by means of cooling or heating.

4. The method recited in claim 3, including the additional steps of monitoring the temperature of the benign tissue area to be protected and the temperature of the targeted tissue area to be treated; and heating or cooling the inflated balloon of the balloon catheter depending upon the monitored temperatures of said protected and treated tissue areas.

5. The method recited in claim 4, including the additional step of displaying the monitored temperatures of the benign tissue area and the targeted tissue area and the difference therebetween.

6. The method recited in claim 4, wherein the step of monitoring the temperatures of the benign tissue area to be protected and the targeted tissue area to be treated includes mounting a pair of temperature sensors on the balloon of the balloon catheter such that one of said temperature sensors is responsive to the temperature of the benign tissue area and the other temperature sensor is responsive to the temperature of the targeted tissue area.

7. The method recited in claim 3, including the additional step of forming a working channel through the balloon catheter such that the balloon surrounds said working channel, and wherein the step of heating or cooling the inflated balloon includes running a supply of heated or cooled liquid through said working channel depending upon whether the targeted tissue area is treated by means of cooling or heating.

8. The method recited in claim 3, including the additional steps of forming a working channel through the balloon catheter such that said balloon surrounds said working channel; and locating a heating wire within said working channel, and wherein the step of heating or cooling the inflated balloon includes heating said heating wire within said working channel when the targeted tissue area is treated by means of cooling.

9. A surgical method for moving a benign internal tissue area of a patient to be protected away from an adjacent targeted internal tissue area to be treated with thermoablative therapy by means of cooling or heating the targeted tissue area to a lethal temperature, said method comprising the steps of:
forming an access channel between the targeted internal tissue area to be treated and the adjacent benign internal tissue area to be protected; and
locating within the access channel a spacer by which to separate the internal tissue area to be protected from the targeted internal tissue area to be treated.

10. The method recited in claim 9, wherein the spacer located in said access channel is a hollow sleeve.

11. The method recited in claim 10, including the additional steps of:
positioning a needle trocar through said hollow sleeve so that said sleeve is carried by said trocar;
moving said needle trocar and said sleeve carried thereby through the tissue of the patient for forming said access channel; and
withdrawing said needle trocar from the patient's tissue leaving said hollow sleeve between the targeted tissue area to be treated and the benign tissue area to be protected.

12. The method recited in claim 11, including the additional step of monitoring the temperature of the benign tissue area to be protected.

13. The method recited in claim 11, including the additional step of monitoring the temperatures of the benign tissue area to be protected and the targeted tissue area to be treated by mounting a pair of temperature sensors on the hollow sleeve such that one of said temperature sensors is responsive to the temperature of the benign tissue area and the other temperature sensor is responsive to the temperature of the targeted tissue area.

14.-28. (canceled)
